(19) United States
(12) Patent Application Publication
(10) Pub. No.: US 2006/0230548 A1
(43) Pub. Date: Oct. 19, 2006

(54) Reductive colorant for keratin fibres

(76) Inventors: Markus Speckbacher, Aschaffcnburg (DE); Hans-Jurgen Braun, Ueberstorf (CH)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/393,079

(22) Filed: Mar. 30, 2006

(30) Foreign Application Priority Data
Apr. 2, 2005 (EP) 05007229.7

(51) Int. Cl. A61K 8/00

(52) U.S. Cl. 8/405

(57) ABSTRACT

Agent for colouring keratin-containing fibres, in particular human hair, comprising a) ascorbic acid, b) at least one oxime compound and c) at least one reactive carbonyl compound, and method of colouring hair using this agent.

The present invention relates to a reductive colouring system consisting of oxime compounds and carbonyl compounds, and ascorbic acid as reducing agent, and to colorants comprising these compounds for keratin fibres, such as, for example, human hair, wool or furs.

In general, two processes are used for colouring keratin-containing fibres. One possibility consists in the use of direct dyes. These are incorporated into suitable carrier masses in order then to be applied to the fibres. This method, generally known as tinting, is easy to use, exceptionally mild and is characterized by low damage to the keratin fibres since no ammonia or peroxide is added. However, the durability and wash resistance of this colouring method is generally unsatisfactory, for which reason a direct method is also referred to as semipermanent hair colour.

A long-lasting coloration, also called permanent hair colour, can be produced with oxidation dyes which are formed by oxidative coupling of one or more developer components with one or more coupler components. If required, oxidation-stable direct dyes can also be added to the oxidative system in order to achieve particular colour effects. When using oxidizing agents, however, damage to the hair structure is observed. Furthermore, some of the oxidation hair colour precursors used (in particular aromatic amines) have a considerable sensitization potential, as a result of which skin irritations may arise in people with a corresponding disposition.

An object of the present invention is therefore to provide a permanent hair colouring system consisting of oxime compounds and carbonyl compounds with which the colorations achieved can on the one hand be applied gently to the fibres, and which is acceptable from a toxicological and dermatological point of view, and on the other hand also permits an intense and brilliant colour result. Furthermore, the resulting hair coloration must have good light fastness and resistance to shampooing (durability of the coloration) and a good rubbing fastness.

Surprisingly, it has now been found that fashionable and luminous colour shades can be produced on keratin-containing fibres if a reductive colorant is applied to the fibres which comprises certain oxime compounds, reactive carbonyl compounds and ascorbic acid (vitamin C). The oxime compounds are reduced to the corresponding amines, which in turn react straightaway with the reactive carbonyl compounds in the hair to give coloured substances. The resulting colorations are extremely resistant to washing and durable. Although of course no hydrogen peroxide (oxidizing agent!) can be used during the reduction process, lightening of the natural colour shade, if this is desired, is possible but not necessarily required in the course of a pretreatment.

The oxime compounds according to the invention can be produced by nitrosation of CH-acidic compounds. CH-acidic compounds are generally regarded as being those compounds which carry a hydrogen atom bonded to an aliphatic carbon atom where, on account of the electron-withdrawing substituents, activation of the corresponding carbon-hydrogen bond is effected. Reactive carbonyl compounds according to the invention have at least one carbonyl group as reactive group.

The present invention therefore provides an agent for colouring keratin-containing fibres, in particular human hair, which is characterized in that it comprises a) ascorbic acid (vitamin C) as reducing agent, b) at least one oxime compound of the formula A1-A23 as “developer substance” and c) at least one reactive carbonyl compound of the formula B1-B17 as “coupler substance”.

The oxime compounds A1-A23 according to the invention are defined as follows:

Compounds with the formula A1: 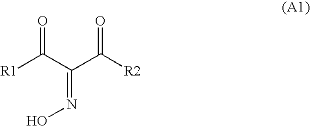
in which R1 is a cyano group, a substituted or unsubstituted aryl group or a substituted or unsubstituted, saturated or unsaturated heterocycle or a group —C—(O)—R3, where R3 is a hydrogen atom or a C₁-C₆-alkyl group; and

R2 is a cyano group, a substituted or unsubstituted Cl-C₆-alkyl group, a C₃-C₆-cycloalkyl group, a substituted or unsubstituted aryl group or a substituted or unsubstituted aminoaryl group or a substituted or unsubstituted, saturated or unsaturated heterocycle.

Compounds with the formula A2: 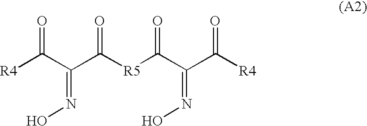
in which R4 is a cyano group, a substituted or unsubstituted aryl group, a substituted or unsubstituted, saturated or unsaturated heterocycle or a group —C—(O)—R6, where R6 is a hydrogen atom or a C₁-C₆-alkyl group; and

R5 is a substituted or unsubstituted C₂-C₆-alkyl group, a C₃-C₆-cycloalkyl group, a substituted or unsubstituted aryl group, a substituted or unsubstituted aminoaryl group, a substituted or unsubstituted, saturated or unsaturated heterocycle.

Compounds with the formulae A3/A4: 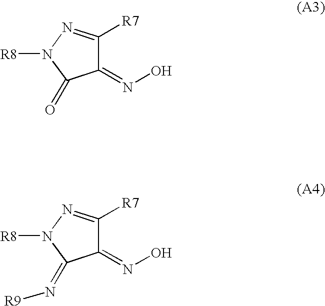
in which R7 and R8 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group, a substituted or unsubstituted aminoaryl group, where R7 can likewise be a C₁-C₆-alkoxy group; and

R9 is a hydrogen atom or a substituted or unsubstituted C₁-C₆-alkyl group.

Compounds with the formula A5: 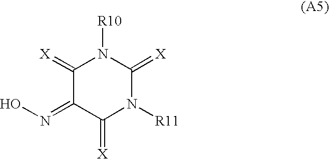
in which R10 and R11 may be identical or different and, independently of one another, are a substituted or unsubstituted aryl group, an aryl-C₁-C₄-alkyl group, a C₁-C₆-alkyl group, a C₃-C₆-cycloalkyl group, a C₂-C₆-alkylene group or a bicycle bonded via the radicals R10 and R11; and X is an oxygen atom or a sulphur atom.

Compounds with the formula A6: 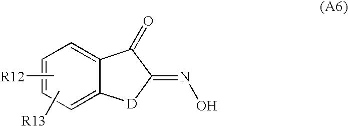
in which D is an oxygen atom, a sulphur atom, a sulphoxyl group, a sulphonyl group or a group N—R₁₂ₐ, where R₁₂ₐ is a hydrogen atom or a substituted or unsubstituted C₁-C₆-alkyl group;

and R12 and R13, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR14R15, where R14 and R15, independently of one another, are a hydrogen atom or a C₁-C₆-alkyl group.

Compounds with the formulae A7/A8: 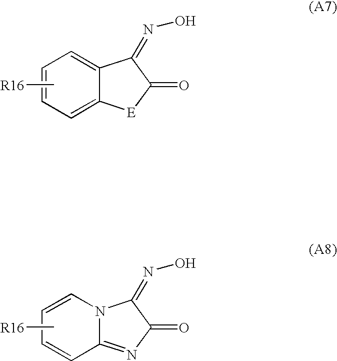
in which E is an oxygen atom, a sulphur atom or a group NR17, where R17 is a hydrogen atom or a substituted or unsubstituted C₁-C₆-alkyl group; and

R16 is a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR18R19, where R18 and R19, independently of one another, are a hydrogen atom or a C₁-C₆-alkyl group.

Compounds with the formula A9: 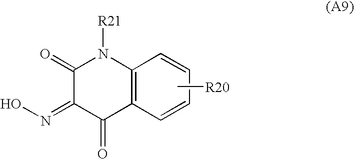
in which R20 is a hydrogen atom, a halogen atom, a hydroxyl group, a nitro group, a C₁-C₆-alkoxy group or a substituted or unsubstituted C₁-C₆-alkyl group; and

R21 is a hydrogen atom or a C₁-C₄-alkyl group.

Compounds of the formulae A10, A11, A12: 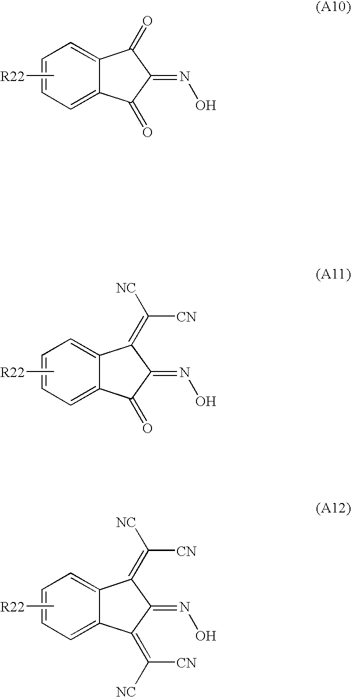
in which R22 is a hydrogen atom, a halogen atom, a hydroxyl group, a nitro group, a C₁-C₆-alkyl group, a C₁-C₆-alkoxy group, a carboxyl group, a carboxamide group, a cyano group or an amino group —NR23R24, where R23 and R24, independently of one another, are a hydrogen atom or a C₁-C₆-alkyl group.

Compounds with the formula A13: 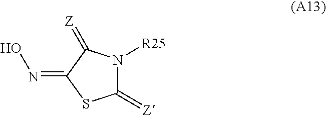
in which Z is an oxygen atom or a group —NR26, where R26 is a hydrogen atom or a C₁-C₆-alkyl group;

Z′ is a sulphur atom or a group —NR27, where R27 is a hydrogen atom or a C₁-C₆-alkyl group; and

R25 is a hydrogen atom, a C₁-C₆-alkyl group or a C₁-C₄-carboxyalkyl group.

Compounds with the formula A14: 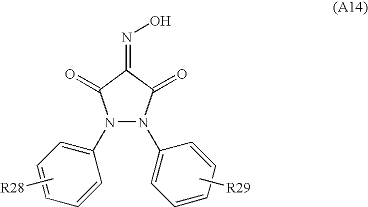
in which R28 and R29 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR30R31, where R30 and R31, independently of one another, are a hydrogen atom or a C₁-C₆-alkyl group.

Compounds with the formula A15: 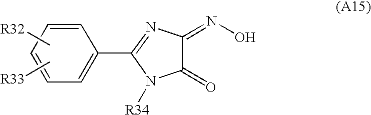
in which R32 and R33 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR35R36, where R35 and R36, independently of one another, are a hydrogen atom or a C₁-C₆-alkyl group; and

R34 is a hydrogen atom or a C₁-C₆-alkyl group.

Compounds with the formula A16: 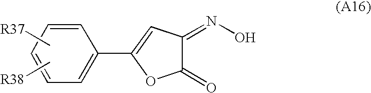
in which R37 and R38 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR39R40, where R39 and R40, independently of one another, are a hydrogen atom or a C₁-C₆-alkyl group.

Compounds with the formulae A17, A18, A19: 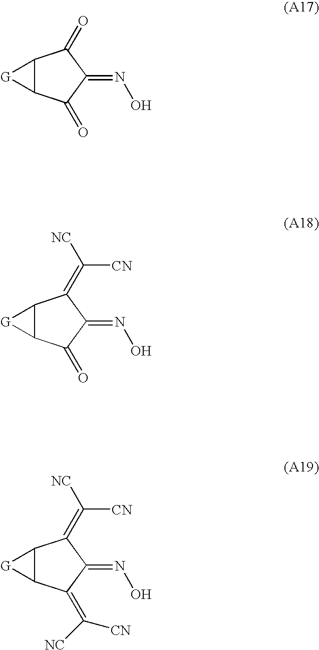
in which G is a fused, substituted or unsubstituted aromatic or heteroaromatic ring to which a further aromatic or heteroaromatic ring may additionally be fused.

Compounds with the formula A20: 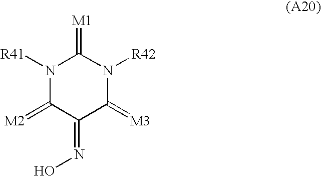
in which R41 and R42 may be identical or different and, independently of one another, are a hydrogen atom or a substituted or unsubstituted C₁-C₆-alkyl group; M1 is an oxygen atom, a sulphur atom or an NH group;

and M2 and M3 may be identical or different and, independently of one another, are an oxygen atom or an NH group.

1,3-Dihydro-2H-inden-2-one oxime with the formula A21:

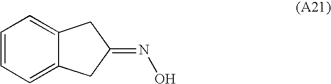

Compounds with the formulae A22, A23: 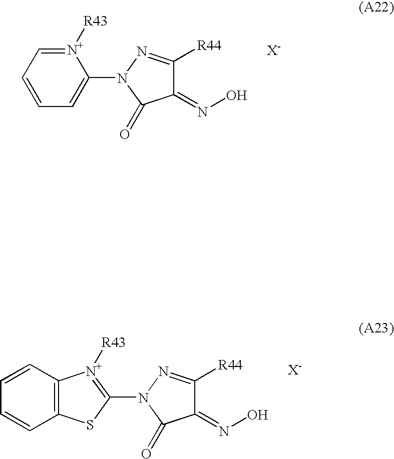
in which R43 is a substituted or unsubstituted C₁-C₆-alkyl group; and R44 is a substituted or unsubstituted aryl group or an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group, a C₁-C₆-alkoxy group or a substituted or unsubstituted aminoaryl group.

The counterions X⁻ used are preferably sulphate anions, rethylsulphate anions, phosphate anions, hydrogenphosphate anions, oxalate anions, formate anions, acetate anions, citrate anions, tartrate anions, malonate anions, pyruvate anions or halogen anions, particular preference being given to the chloride anion, bromide anion and methylsulphate anion.

The oxime compounds (developer substances) of the general formula A1-A23 are preferably chosen from diethyl 2-(hydroxyimino)-malonate, 1H-indene-1,2,3-trione 2-oxime, (2Z)-5-(dimethylamino)-1H-indene-1,2,3-trione 2-oxime, (2Z)-4,5-dimethoxy-1H-indene-1,2,3-trione 2-oxime, (2Z)-5-methoxy-1H-indene-1,2,3-trione 2-oxime, (2Z)-5-nitro-1H-indene-1,2,3-trione 2-oxime, (2Z)-4,6-dichloro-1H-indene-1,2,3-trione 2-oxime, (6Z)-5H-cyclo-penta[c]pyridine-5,6,7-trione 6-oxime, (6Z)-5H-cyclopenta[b]pyridine-5,6,7-trione 6-oxime, (2E)-1H-cyclopenta[a]naphthalene-1,2,3-trione 2-oxime, 1H-cyclo-penta[b]quinoxaline-1,2,3-trione 2-oxime, (2Z)4-methyl-4,8b-dihydrocyclopenta[b]indole-1,2,3(3aH)-trione 2-oxime, (5E)-4H-cyclopenta[b]thiophene-4,5,6-trione 5-oxime, (2Z)-5-methyl-1H-indene-1,2,3-trione-2-oxime, (2E)-4,6-dichloro-1H-indene-1,2,3-trione 2-oxime, 4,7-dichloro-1H-indene-1,2,3-trione 2-oxime, (2Z)-1H-cyclopenta[b]quinoline-1,2,3-trione 2-oxime, (2Z)-1-benzothiophene-2,3-dione 2-oxime 1,1-dioxide, (2Z)-1-benzothio-phene-2,3-dione 2-oxime, (2Z)-1-benzofuran-2,3-dione 2-oxime, (3E)-imidazo[1,2-a]pyridine-2,3-dione 3-oxime, (3Z)-1H-indole-2,3-dione 3-oxime, (3Z)-1-methyl-1H-indole-2,3-dione 3-oxime, (3Z)-6-chloro-1-methyl-1H-indole-2,3-dione 3-oxime, (3E)-7-chloroimidazo[1,2-a]pyridine-2,3-dione 3-oxime, 1,2-diphenyl-3,4,5-pyrazolidinetrione-4-oxime, 1H-cyclopenta[b]naphthalene-1,2,3-trione 2-oxime, 2-[(2E)-2-(hydroxyimino)-3-oxo-2,3-dihydro-1H-indene-1-ylidene]malononitrile, (4Z)-3-methyl-1-phenyl-1H-pyrazole-4,5-dione 4-oxime, 1,3-diethyl-2-thioxodihydro-4,5,6(1H)-pyrimidinetrione 5-oxime, (5Z)-2-thioxo-1,3-thiazolidine-4,5-dione 5-oxime, (4Z)-3-methyl-1-(4-nitrophenyl)-1H-pyrazole-4,5-dione 4-oxime, 1,3-dihydro-2H-inden-2-one oxime, 4,5,6,7-tetrachloro-1H-indene-1,2,3-trione 2-oxime, 2-[(4E)-4-(hydroxyimino)-3-methyl-5-oxo-4,5-dihydro-1H-pyrazol-1-yl]-1-methylpyridinium methylsulphate and 2-[(4E)-4-(hydroxyimino)-3-methyl-5-oxo-4,5-dihydro-1H-pyrazol-1-yl]-3-methyl-1,3-benzothiazol-3-ium methylsulphate.

The carbonyl compounds B1-B16 according to the invention are defined as follows:

Compounds with the formula B1: 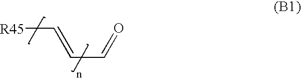
in which R45 is a substituted or unsubstituted aryl group to which a further aromatic ring may optionally also be fused, or a substituted or unsubstituted, saturated or unsaturated heterocycle; and n is 0, 1, 2 or 3.

Compounds with the formula B2: 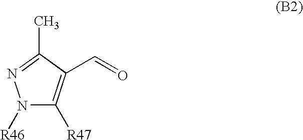
in which R46 is a C₁-C₆-alkyl group, a substituted or unsubstituted aryl radical or a substituted or unsubstituted, saturated or unsaturated heterocycle; and

R47 is a pyrrole radical, an imidazole radical, a pyrazole radical, an indole radical, a pyrrolidine radical, a morpholine radical, a dimethylamine radical, a phenol radical or a thiophenol radical, where these radicals are in each case bonded to the general formula B2 via the heteroatom.

Pyridoxal (vitamin B₆) with the formula B3:

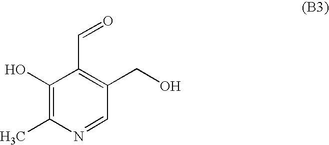

All-trans-retinal (vitamin A) with the formula B4:

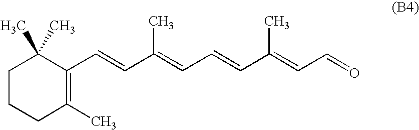

Compounds with the formula B5: 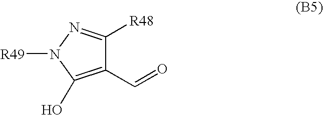
in which R48 and R49 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group or a substituted or unsubstituted aminoaryl group, and R48 can likewise be a C₁-C₆-alkoxy group.

Compound with the formula B6: 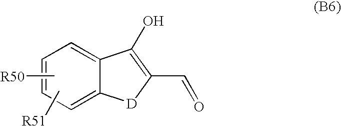
in which D has the abovementioned meaning; and

R50 and R51 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR52R53, where R52 and R53, independently of one another, are a hydrogen atom or a C₁-C₆-alkyl group.

Compounds with the formulae B7/B8: 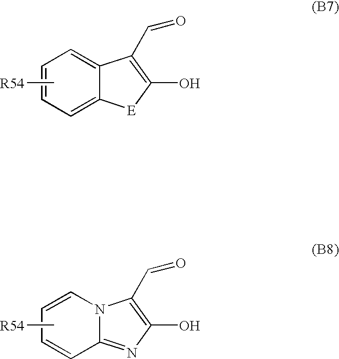
in which E has the abovementioned meaning; and

R54 is a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR55R56, where R55 and R56, independently of one another, are a hydrogen atom or a C₁-C₆-alkyl group.

Compounds with the formula B9: 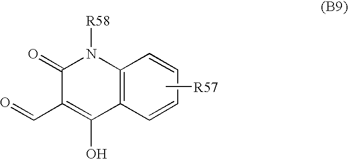
in which R57 is a hydrogen atom, a halogen atom, a hydroxyl group, a nitro group, a C₁-C₆-alkoxy group or a substituted or unsubstituted C₁-C₆-alkyl group; and

R58 is a hydrogen atom or a C₁-C₄-alkyl group.

Compounds with the formulae B10/B11: 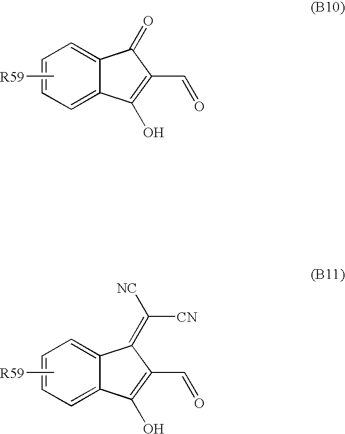
in which R59 is a hydrogen atom, a halogen atom, a hydroxyl group, a nitro group, a C₁-C₆-alkyl group, a C₁-C₆-alkoxy group, a carboxyl group, a carboxamide group, a cyano group or an amino group —NR60R61, where R60 and R61, independently of one another, are a hydrogen atom or a C₁-C₆-alkyl group.

Compounds with the formula B12: 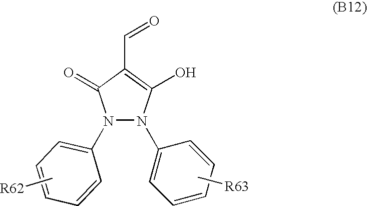
in which R62 and R63 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR64R65, where R64 and R65, independently of one another, are a hydrogen atom or a C₁-C₆-alkyl group.

Compounds with the formulae B13/B14: 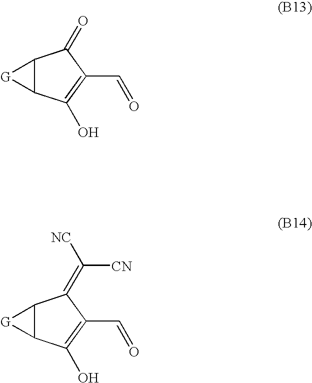
in which G has the abovementioned meaning.

Compounds with the formulae B15/B16: 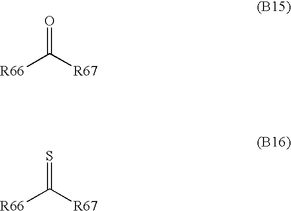
in which R66 and R67 may be identical or different and, independently of one another, are a C₁-C₆-alkyl group or a substituted or unsubstituted aryl group.

Compounds with the formula B17: 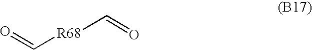
in which R68 is a substituted or unsubstituted C₁-C₆-alkyl group, a C₁-C₆-alkylene group or a substituted or unsubstituted aryl group.

The reactive carbonyl compounds (coupler substances) of the general formulae B1-B17 are preferably chosen from benzaldehyde, 4-methoxybenzaldehyde, 4-hydroxybenzaldehyde, 3,4-dimethoxybenzaldehyde, 2,5-dimethoxybenzaldehyde, 3-hydroxy-4-methoxybenzaldehyde, 4-nitrobenzaldehyde, 4-hydroxy-3-methoxybenzaldehyde, 4-(dimethylamino)benzaldehyde, (2E)-3-[4-(dimethylamino)phenyl]-2-propenal, 2,4,6-trihydroxybenzaldehyde, 3-hydroxy-5-(hydroxymethyl)-2-methylisonicotine aldehyde, all-trans-retinal, 3,5-dioxo-1,2-diphenyl-4-pyrazolidinecarbaldehyde, (2E,4E)-5-phenyl-2,4-pentadienal, pentanedial, 4-(methylsulphanyl)benzaldehyde, 1-naphthaldehyde, (2E)-3-(4-nitrophenyl)-2-propenal, 3-phenoxybenzaldehyde, terephthalaldehyde, 3-hydroxy-1-oxo-1H-cyclopenta[a]-naphthalene-2-carbaldehyde, 3-hydroxy-1-oxo-1H-cyclopenta[b]-quinoxaline-2-carbaldehyde, 3-hydroxy-4-methyl-1-oxo-1,3a,4,8b-tetra-hydrocyclopenta[b]indole-2-carbaldehyde, 4-hydroxy-6-oxo-6H-cyclopenta[b]thiophene-5-carbaldehyde, 3-hydroxy-5-nitro-1-oxo-1H-indene-2-carbaldehyde, 4,6-dichloro-3-hydroxy-1-oxo-1H-indene-2-carbaldehyde, 7-hydroxy-5-oxo-5H-cyclopenta[c]pyridine-6-carbaldehyde, 7-hydroxy-5-oxo-5H-cyclopenta[b]pyridine-6-carbaldehyde, 3-hydroxy-1-oxo-1H-indene-2-carbaldehyde, 5-(dimethylamino)-3-hydroxy-1-oxo-1H-indene-2-carbaldehyde, 3-hydroxy-4,5-dimethoxy-1-oxo-1H-indene-2-carbaldehyde, 3-hydroxy-5-methoxy-1-oxo-1H-indene-2-carbaldehyde, 3-methyl-5-(4-morpholinyl)-1-phenyl-1H-pyrazole-4-carbaldehyde, 3-methyl-1-phenyl-5-(1H-pyrrol-1-yl)-1H-pyrazole-4-carbaldehyde, 3-methyl-1-phenyl-5-(1-pyrrolidinyl)-1H-pyrazol-4-carbaldehyde, 3-methyl-1-(2-pyridinyl)-5-(1-pyrrolidinyl)-1H-pyrazole-4-carbaldehyde, 3-methyl-1-(2-pyridinyl)-5-(1H-pyrrol-1-yl)-1H-pyrazole-4-carbaldehyde, 4-methoxy-1-naphthaldehyde, 1H-indole-3-carbaldehyde, bis[4-(dimethylamino)-phenyl]methanethione, 2,4-dihydroxybenzaldehyde, 1-methyl-1H-indole-3-carbaldehyde and 3,4-dihydroxybenzaldehyde.

The oxime compounds according to the invention of the general formula A1-A23 can be prepared by nitrosation of the CH-acidic starting compounds. These are either available commercially or are accessible through standard operations from components which are commercially available or can be prepared easily. Thus, for example, the corresponding oxime compound can be prepared from 1,3-indanedione with sodium nitrite in hydrochloric solution (scheme 1).

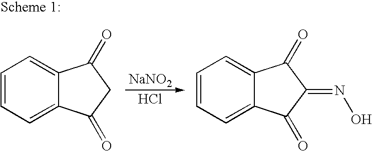

Substituted 1,3-indanediones can be prepared, for example, in accordance with information in the literature (G. Sartori et al., J. Chem. Soc. Perkin. Trans. 1, 1992, 2985-2988) by Friedel-Crafts cycloacylation (cross condensation) from aromatic carbonyl chlorides, anhydrous aluminium chloride and malonyl dichloride (scheme 2).

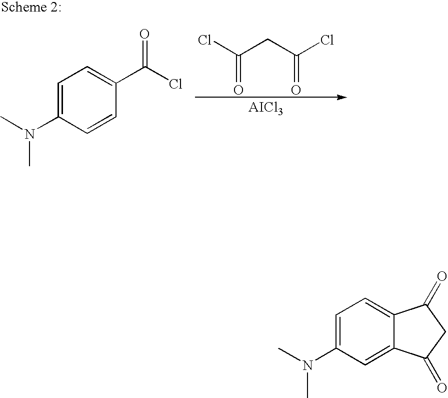

The oxime compounds resulting therefrom can be prepared by nitrosation of the substituted indanedione derivatives with neopentyl nitrite in solvents such as, for example, acetonitrile.

Aromatic and heteroaromatic aldehydes, bisaldehydes and ketones (reactive carbonyl compounds of the general formulae B1-B17) are commercially available in large numbers or obtainable from standard reactions. Carbaldehydes are likewise accessible by standard operations from the corresponding CH-acidic compounds.

1,3-Indanedione-2-carbaldehyde can be readily prepared, for example, in accordance with Gudrinietse et al. (Zhurnal Organicheskoi Khimii, Vol. 9, No. 2, 1973, 336-338) by an aldol condensation of 1,3-indanedione with chloral and subsequent alkaline hydrolysis (scheme 3).

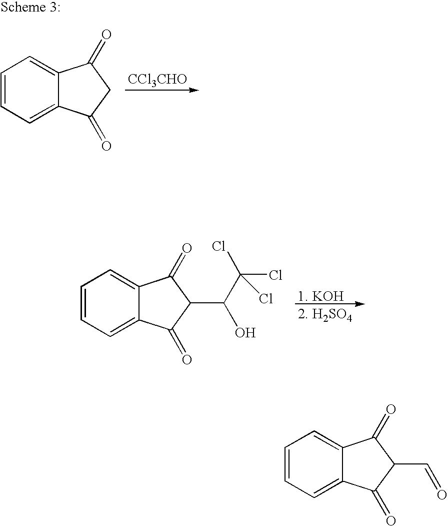

A large number of carbaldehydes can be prepared analogously to this reaction from various CH-acidic compounds.

During the colouring process, the oxime compounds according to the invention of the general formula A1-A23 are reduced to give the corresponding amines, which, in the hair, react very rapidly with the carbonyl compounds according to the invention of the general formula B1-B17 to give the corresponding coloured compounds (azomethines or Schiff's bases) (scheme 4).

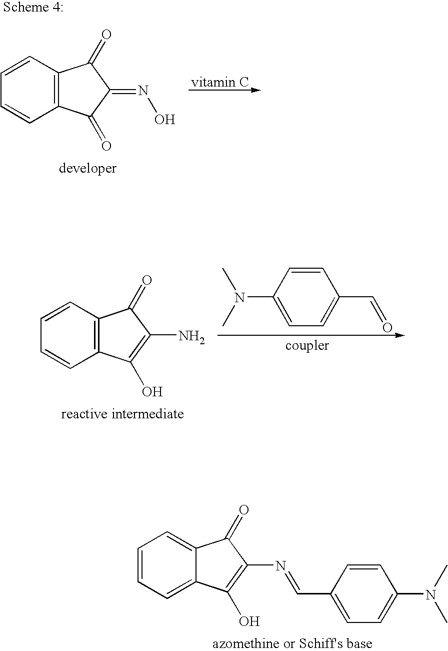

The oxime compounds according to the invention of the general formula A1-A23 and the carbonyl compounds according to the invention of the general formula B1-B17 are present in the colorants according to the invention preferably in a total amount of from 0.01 to 10 per cent by weight, in particular 0.1 to 8 per cent by weight.

To produce special colour nuances, besides the components used according to the invention, it is possible to additionally also add to the agents aording to the invention one or more customary direct dyes from the group of acidic and basic dyes, nitro dyes, azo dyes, anthraquinone dyes and triphenylmethane dyes, such as for example, 1,4-bis[(2-hydroxyethyl)amino]-2-nitrobenzene, 1-(2-hydroxyethyl)amino-2-nitro-4-[di-(2-hydroxyethyl)amino]benzene, (HC Blue No. 2), 1-amino-3-methyl4-[(2-hydroxyethyl)amino]-6-nitrobenzene, (HC Violet No. 1), 4-[ethyl-(2-hydroxyethyl)amino]-1-[(2-hydroxyethyl)amino]-2-nitrobenzene hydrochloride (HC Blue No. 12), 1-[(2,3-dihydroxypropyl)amino]-4-[methyl-(2-hydroxyethyl)amino]-2-nitrobenzene (HC Blue No. 10), 1-[(2,3-dihydroxypropyl)amino]-4-[ethyl-(2-hydroxyethyl)amino]-2-nitrobenzene hydrochloride (HC Blue No. 9), 1-(3-hydroxypropylamino)-4-[di-(2-hydroxyethyl)amino]-2-nitrobenzene, (HC Violet No. 2); 1-amino-4-[(2-hydroxyethyl)amino]-2-nitrobenzene (HC Red No. 7), 2-amino-4,6-dinitrophenol, 1,4-diamino-2-nitrobenzene (CI76070), 4-amino-2-nitrodiphenylamine (HC Red No. 1), 1-amino-4-[di(2-hydroxyethyl)amino]-2-nitrobenzene hydrochloride (HC Red No. 13), 1-amino-5-chloro-4-[(2-hydroxyethyl)amino]-2-nitrobenzene, 4-amino-1-[(2-hydroxyethyl)amino]-2-nitrobenzene (HC Red No. 3), 4-amino-2-nitro-1-((prop-2-en-1-yl)amino)benzene, 4-amino-3-nitrophenol, 4-[(2-hydroxyethyl)amino]-3-nitrophenol, 4-[(2-nitrophenyl)amino]phenol (HC Orange No. 1), 1-[(2-aminoethyl)amino]-4-(2-hydroxyethoxy)-2-nitrobenzene (HC Orange No. 2), 4-(2,3-dihydroxypropoxy)-1-[(2-hydroxyethyl)amino]-2-nitrobenzene, (HC Orange No. 3), 1-amino-5-chloro-4-[(2,3-dihydroxypropyl)amino]-2-nitrobenzene (HC Red No. 10), 5-chloro-1,4-[di(2,3-dihydroxypropyl)amino]-2-nitrobenzene (HC Red No. 11), 2-[(2-hydroxyethyl)amino]-4,6-dinitrophenol, 4-ethylamino-3-nitrobenzoic acid, 2-[(4-amino-2-nitrophenyl)amino]benzoic acid, 2-chloro-6-ethylamino-4-nitrophenol, 2-amino-6-chloro-4-nitrophenol, 4-[(3-hydroxypropyl)amino]-3-nitrophenol, 2,5-diamino-6-nitropyridine, 3-amino-6-(methylamino)-2-nitropyridine, 1,2,3,4-tetrahydro-6-nitroquinoxaline, 7-amino-3,4-dihydro-6-nitro-2H-1 ,4-benzoxazine (HC Red No. 14), 1,2-diamino-4-nitrovenzene (CI76020), 1-amino-2-[(2-hydroxyethyl)amino]-5-nitrobenzene (HC Yellow No. 5), 1-(2-hydroxyethoxy)-2-[(2-hydroxyethyl)amino]-5-nitrobenzene, (HC Yellow No. 4), 1-[(2-hydroxyethyl)amino]-2-nitrobenzene (HC Yellow No. 2), 2-[(2-hydroxyethyl)amino]-1-methoxy-5-nitrobenzene, 2-amino-3-nitro-phenol, 1-amino-2-methyl-6-nitrobenzene, 1-(2-hydroxyethoxy)-3-methylamino-4-nitrobenzene, 2,3-(dihydroxypropoxy)-3-methylamino-4-nitrobenzene, 2-[(2-hydroxy-ethyl)amino]-5-nitrophenol (HC Yellow No. 11), 3-[(2-aminoethyl)amino]-1-methoxy-4-nitrobenzene hydrochloride, (HC Yellow No. 9), 1-[(2-ureidoethyl)amino]-4-nitrobenzene, 4-[(2,3-dihydroxypropyl)amino]-3-nitro-1-trifluoromethylbenzene, (HC Yellow No. 6), 1-chloro-2,4-bis[(2-hydroxyethyl)amino]-5-nitrobenzene (HC Yellow No. 10), 4-[(2-hydroxyethyl)amino]-3-nitro-1-methylbenzene, 1-chloro-4-[(2-hydroxyethyl)amino]-3-nitrobenzene (HC Yellow No. 12), 4-[(2-hydroxyethyl)amino]-3-nitro-1-trifluoromethylbenzene, (HC Yellow No. 13), 4-[(2-hydroxyethyl)amino]-3-nitrobenzonitrile (HC Yellow No. 14), 4-[(2-hydroxyethyl)amino]-3-nitrobenzamide (HC Yellow No. 15), 2,4-dinitro-1-hydroxynaphthalene, 1,4-di[(2,3-dihydroxypropyl)amino]-9,10-anthraquinone, 1,4-di[(2-hydroxyethyl)amino]-9,10-anthraquinone (CI61545, Disperse Blue 23), 1-amino-4-hydroxy-9,10-anthraquinone (CI607 10, Disperse Red 15), 1-hydroxy-4-[(4-methyl-2-sulphophenyl)-amino]-9,10-anthraquinone, 7-beta-D-glucopyranosyl-9,10-dihydro-1-methyl-9,10-dioxo-3,5,6,8-tetrahydroxy-2-anthracenecarboxylic acid (CI75470, Natural Red 4), 1-[(3-aminopropyl)amino]-9,10-anthraquinone (HC Red No. 8), 1,4-diamino-9,10-anthraquinone (CI61 100, Disperse Violet No. 1), 1-amino-4-(methylarnino)-9,10-anthraquinone (CI61105, Disperse Violet No. 4, Solvent Violet No. 12), N-(6-((3-chloro-4-(methylamino)phenyl)imino)-4-methyl-3-oxo-1,4-cyclohexadien-1-yl)urea (HC Red No. 9), 2-((4-(di(2-hydroxyethyl)-amino)phenyl)amino)-5-((2-hydroxyethyl)-amino)-2,5-cyclohexadiene-1,4-dione (HC Green No. 1), 2-hydroxy-1,4-naphthoquinone (CI75480, Natural Orange No. 6), 1,2-dihydro-2-(1,3-dihydro-3-oxo-2H-indol-2-ylidene)-3H-indol-3-one (CI73000), 1,3-bis(dicyanomethylene)indane, di[4-(diethylamino)phenyl]-[4-(ethylamino)-naphthyl]carbenium chloride (CI42595; Basic Blue No. 7), di[4-(dimethylamino)phenyl][4-(phenylarnino)naphthyl]carbenium chloride (CI44045; Basic Blue No. 26), Basic Blue No. 77, 8-amino-2-bromo-5-hydroxy-4-imino-6-[(3-(trimethylammonio)phenyl)amino]-1(4H)-naphthalinone chloride (CI56059; Basic Blue No. 99) tri(4-amino-3-methylphenyl)carbenium chloride (CI42520; Basic Violet No. 2), di(4-aminophenyl)(4-amino-3-methylphenyl)carbenium chloride (CI42510; Basic Violet No. 14), 1-[(4-aminophenyl)azo]-7-(trimethylarnmonio)-2-naphthol chloride (CI12250; Basic Brown No. 16), 3-[(4-amino-2,5-dimethoxyphenyl)azo]-N,N,N-trimethyl-benzenaminium chloride (CI112605, Basic Orange No. 69), 1-[(4-amino-2-nitrophenyl)azo]-7-(trimethylammonio)-2-naphthol chloride (Basic Brown No. 17), 1-[(4-amino-3-nitrophenyl)azo]-7-(trimethylammonio)-2-naphthol chloride (CI12251; Basic Brown No. 17), 2-((4-aminophenyl)azo)-1,3-dimethyl-1H-imidazol-3-ium chloride (Basic Orange No. 31), 3,7-diamino-2,8-dimethyl-5-phenylphenazinium chloride (CI50240; Basic Red No. 2), 1,4-dimethyl-5-[(4-(dimethylamino)phenyl)azo]-1,2,4-triazolium chloride (CI1055; Basic Red No. 22), 1,3-dimethyl-2-((4-dimethylamino)phenyl)azo-1H-imidazol-3-ium chloride (Basic Red No. 51), 2-hydroxy-1-[(2-methoxyphenyl)azo]-7-(trimethylammonio)naphthalene chloride (CI12245; Basic Red No. 76),

3-methyl-1-phenyl-4-[(3-(trimethylammonio)phenyl)azo]-pyrazol-5-one chloride (CI12719; Basic Yellow No. 57), 1-methyl-4-((methylphenylhydrazono)methyl)pyridinium methylsulphate (Basic Yellow No. 87), 1-(2-morpholiniumpropylamino)-4-hydroxy-9,10-anthraquinone methylsulphate, 1-[(3-(dimethylpropylaminium)propyl)amino]-4-(methylamino)-9,10-anthraquinone chloride, 1-[di(2-hydroxyethyl)amino]-3-methyl-4-[(4-nitrophenyl)azo]benzene (CI11210, Disperse Red No. 17), 1-[di(2-hydroxyethyl)amino]-4-[(4-nitrophenyl)azo]benzene, (Disperse Black No. 9), 4-[(4-aminophenyl)azo]-1-[di(2-hydroxyethyl)amino]-3-methylbenzene, (HC Yellow No. 7), 2,6-diamino-3-[(pyridin-3-yl)azo]pyridine and 2-((4-(ethyl(2-hydroxyethyl)amino)-2-methylphenyl)azo)-5-nitro-1,3-thiazole (CI111935; Disperse Blue No. 106), 3-(2′,6′-diamino-pyridyl-3′-azo)pyridine (=2,6-diamino-3-((pyridin-3-yl)azo)pyridine, N,N-di(2-hydroxyethyl)-3-methyl-4-((4-nitrophenyl)azo)aniline (Disperse Red 17, CI 11210), 3-diethylamino-7-(4-dimethylaminophenylazo)-5-phenylphenazinium chloride (CI 11050), 4-(2-thiazolylazo)resorcinol, 4-((4-phenylamino)azo)benzosulphonic acid sodium salt (Orange IV), 1-((3-aminopropyl)amino)-9,10-anthracenedione, (HC Red No. 8), 3′,3″,4,5,5′,5″,6,7-octabromophenol sulphonephthalein (Tetrabromophenol Blue). 1-((4-amino-3,5-dimethylphenyl)-(2,6-dichlorophenyl)methylene)-3,5-dimethyl-4-imino-2,5-cyclohexadiene phosphoric acid (1:1) (Basic Blue 77), 3′,3″,5′,5″-tetrabromo-m-cresol sulphonephthalein, 2,4-dinitro-1-naphthol-7-sulphonic acid disodium salt (Acid Yellow 1, CI 10316),

4-[2′-hydroxy-1′-naphthyl)azo]benzosulphonic acid sodium salt (Acid Orange 7, CI 15510), 3′,6′-dihydroxy-2′,4′,5′,7′-tetraiodospiro-[isobenzofuran-1(3H), 9′(9H)-xanthen]-3-one disodium salt (Acid Red 51, CI 45430), 6-hydroxy-5-((2-methoxy-5-methyl-4-sulphophenyl)azo)-2-naphthalenesulphonic acid disodium salt (FD&C Red 40, CI 16035),

2,4-dinitro-1-naphthol sodium salt (Acid Yellow 24; Cl 10315), 2′,4′,5′,7′-tetrabromo-4,5,6,7-tetrachloro-3′,6′-dihydroxyspiro-(isobenzofuran-1(3H), 9′[9H]xanthen]-3-one disodium salt (Acid Red 92; CI 45410), 4-(2-hydroxy-1-naphthylazo)-3-methyl-benzenesulphonic acid sodium salt (Acid Orange 8, CI 15575), 2-amino-1,4-naphthalenedione, dithizone (1,5-diphenylthiocarbazone), N-(2-hydroxyethyl))-2-nitro-4-trifluoromethyl)aniline (HC Yellow 13), N-(2-hydroxyethyl)-4-nitroaniline and 4-chloro-N-(2,3-dihydroxypropyl)-2-nitroaniline.

The abovementioned direct dyes may be present in a total amount of from about 0.01 to 4 per cent by weight, where the total content of dyes in the colorant according to the invention is preferably about 0.01 to 10 per cent by weight, in particular 0.1 to 5 per cent weight.

In addition, the colorants according to the invention can also comprise naturally occurring dyes, such as, for example, henna red, henna neutral, henna black, camomile, sandalwood, black tea, buckthorn bark, sage, logwood, madder root, catechu, sedre and alkanna root.

The colorants according to the invention produce intense colorations even at physiologically compatible temperatures of less than 45° C. They are therefore particularly suitable for colouring human hair. For use on human hair, the colorants are usually incorporated into a hydrous cosmetic carrier. Suitable cosmetic carriers are, for example. creams. emulsions, gels or else surfactant-containing foaming solutions, such as, for example, shampoos or other preparations which are suitable for application to keratin-containing fibres. If necessary, it is also possible to incorporate the colorants into anhydrous carriers, powders, pellets or granules.

The colorant according to the invention can further comprise all additives which are customary and known for such preparations, for example perfume oils, complexing agents, waxes, preservatives, thickeners, antioxidants, alginates, guar gum, haircare substances, such as, for example, cationic polymers or lanolin derivatives, or anionic, nonionic, zwitterionic, amphoteric or cationic surface-active substances (surfactants). Preferably, amphoteric or nonionic surface-active substances are used, for example betaine surfactants, propionates and glycinates, such as, for example, cocoamphoglycinates or cocoamphodiglycinates, ethoxylated surfactants with 1 to 1000 ethylene oxide units, preferably with 1 to 300 ethylene oxide units, such as, for example, glyceride alkoxylates, for example castor oil ethoxylated with 25 ethylene oxide units, polyglycolamides, ethoxylated alcohols and ethoxylated fatty alcohols (fatty alcohol alkoxylates) and ethoxylated fatty acid sugar esters, in particular ethoxylated sorbitan fatty acid esters. The abovementioned constituents are used in the amounts customary for such purposes, for example the surface-active substances in a concentration of from 0.1 to 30 per cent by weight, and the care substances in an amount of from 0.1 to 5 per cent by weight.

The colorant according to the invention can, particularly if it is a hair colorant, be in the form of a powder or of granules, which is/are dissolved prior to application in an aqueous or aqueous-alcoholic preparation, or in the form of an aqueous or aqueous-alcoholic solution, a cream, a gel, an emulsion or an aerosol foam, where the hair colorant can be formulated either in the form of a single-component preparation or else in the form of a multicomponent preparation, for example in the form of a two-component preparation, in which case the particular oxime derivatives of the general formula A1-A23 and the corresponding carbonyl compounds of the general formulae B1-B17 are packaged separately from the other constituents (e.g., the ascorbic acid) and the ready-to-use hair colorant is only prepared immediately prior to application by mixing the two components.

The colorant according to the invention generally has a pH of from about 2 to 6, preferably about 3 to 5. Both organic and also inorganic acids are suitable for adjusting the pH according to the invention. Examples of suitable acids are the following acids: α-hydroxycarboxylic acids, such as, for example, glycolic acid, lactic acid, tartaric acid, citric acid or malic acid, gluconolactone, acetic acid, hydrochloric acid or phosphoric acid, and mixtures of these acids. Particular preference here is given to the use of the ascorbic acid used as reducing agent.

The colorant according to the invention is generally used by applying to the hair an amount of the hair colorant adequate for the hair colouring, about 30 to 120 grams depending on the length of hair, leaving the hair colorant to act at about 15 to 45 degrees Celsius for about 1 to 60 minutes, preferably 5 to 30 minutes, then thoroughly rinsing the hair with water, optionally washing with a shampoo and/or after-treating with a hair-conditioning composition and finally drying.

The colorant described above can also comprise natural or synthetic polymers or modified polymers of natural origin customary for cosmetic compositions, through which setting of the hair is achieved at the same time as the colouring. Such compositions are generally referred to as tinting setting compositions or colour setting compositions.

Of the synthetic polymers known for this purpose in cosmetics, mention may be made, for example, of polyvinylpyrrolidone, polyvinyl acetate, polyvinyl alcohol or polyacrylic compounds, such as polyacrylic acid or polymethacrylic acid, basic polymers of esters of polyacrylic acid, polymethacrylic acid and aminoalcohols, for example their salts or quatemization products, polyacrylonitrile, polyvinyl acetates and copolymers of such compounds, such as, for example, polyvinylpyrrolidone-vinyl acetate; whereas natural polymers or modified natural polymers which may be used are, for example, chitosan (deacetylated chitin) or chitosan derivatives. The abovementioned polymers may be present in the colorant according to the invention in the amounts customary for such compositions, in particular in an amount of from about 1 to 5 per cent by weight.

The hair colorant with additional setting is used in a known and customary manner by wetting the hair with the setting composition, fixing (arranging) the hair in the hairstyle and then drying.

The colorant according to the invention permits an even, intense and long-lasting coloration of keratin fibres (for example human hair, wool or furs) without noteworthy discoloration of the skin and/or scalp.

The examples below are intended to illustrate the subject-matter of the invention in more detail without limiting it thereto.

### EXAMPLES

### Example 1

### Synthesis of 2-[(2E)-2-(hydroxyimino)-3-oxo-2,3-dihydro-1H-inden-1-ylidene]malononitrile (b)

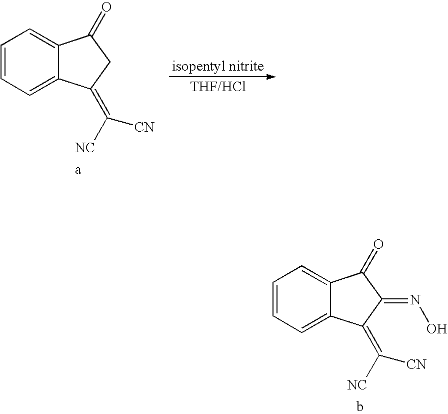

0.20 g (1.03 mmol) of 2-(3-oxo-2,3-dihydro-1H-inden-1-ylidene)-malononitrile (a) are dissolved in 10 ml of tetrahydrofuran (THF). This produces a clear green solution which is cooled to 0° C. 0.35 ml (4.12 mmol) of 37% hydrochloric acid are then added. A colour change from green to orange is observed. 0.14 ml (1.03 mmol) of isopentyl nitrite are now added dropwise to the cooled solution, which is stirred for a further 30 minutes at 0° C. until a clear yellow solution has formed. This is left to stand overnight at room temperature and the resulting precipitate is removed by suction. The product (b) is isolated as white powder in a yield of 44%.

¹H NMR (d₆-DMSO/300 MHz): δ=7.42 (s, 4H, aromatic).

### Examples 2 to 18

### Hair Colorants

| | | |
|---|---|---|
| 5 | mmol | oxime compound of the general formula A1-A23 |
| 5 | mmol | carbonyl compounds of the general formula B1-B17 |
| 5.0 | g | ethanol |
| 4.0 | g | decylpolyglucose |
| 0.2 | g | ethylenediaminotetraacetic acid disodium salt hydrate |
| ad 100.0 | g | water, demineralized |

The hair colouring is carried out by applying an amount of the colorant and of the reducing agent ascorbic acid (preferably 1-4 g/10 ml of colouring solution) adequate for the hair colouring to the hair.

After a contact time of 30 minutes at 40° C., the hair is rinsed with lukewarm water and dried.

The colouring results are summarized in Tables 1, 2 and 3 below.

Unless otherwise indicated, all percentages are by weight.

TABLE 1
Oxime compound: 1H-indene-1,2,3-trione 2-oxime

| | | Colouring |
|---|---|---|
| Ex. No. | Carbonyl compound | result |
| 2 | 4-Hydroxy-3-methoxybenzaldehyde | golden brown |
| 3 | 4-(Dimethylamino)benzaldehyde | rust red |
| 4 | 4-Methoxy-1-naphthaldehyde | golden brown |
| 5 | (2E)-3-[4-Dimethylamino)phenyl]-2-propenal | dark red |
| 6 | 1H-indole-3-carbaldehyde | golden yellow |
| 7 | Bis[4-(dimethylamino)phenyl]-methanethione | pale brown |
| 8 | 2,4-Dihydroxybenzaldehyde | golden yellow |
| 9 | 2,4,6-Trihydroxybenzaldehyde | orange yellow |
| 10 | 3-Hydroxy-4-methoxybenzaldehyde | golden brown |

TABLE 2
Oxime compound:
(4Z)-3-Methyl-1-phenyl-1H-pyrazole-4,5-dione 4-oxime

| | | Colouring |
|---|---|---|
| Ex. No. | Carbonyl compound | result |
| 11 | 4-Hydroxy-3-methoxybenzaldehyde | yellow orange |
| 12 | 4-(Dimethylamino)benzaldehyde | orange |
| 13 | 1-Methyl-1H-indole-3-carbaldehyde | yellow orange |
| 14 | (2E)-3-[4-Dimethylamino)phenyl]-2-propenal | garnet red |
| 15 | 3,4-Dihydroxybenzaldehyde | golden orange |

TABLE 3
Oxime compound: 1,3-Dihydro-2H-inden-2-one oxime

| | | Colouring |
|---|---|---|
| Ex. No. | Coupler | result |
| 16 | 4-Hydroxy-3-methoxybenzaldehyde | lemon yellow |
| 17 | 4-(Dimethylamino)benzaldehyde | lemon yellow |
| 18 | (2E)-3-[4-Dimethylamino)phenyl]-2-propenal | orange |

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. An agent for colouring keratin-containing fibres, comprising a) ascorbic acid, b) at least one oxime compound of the formula A1-A23; 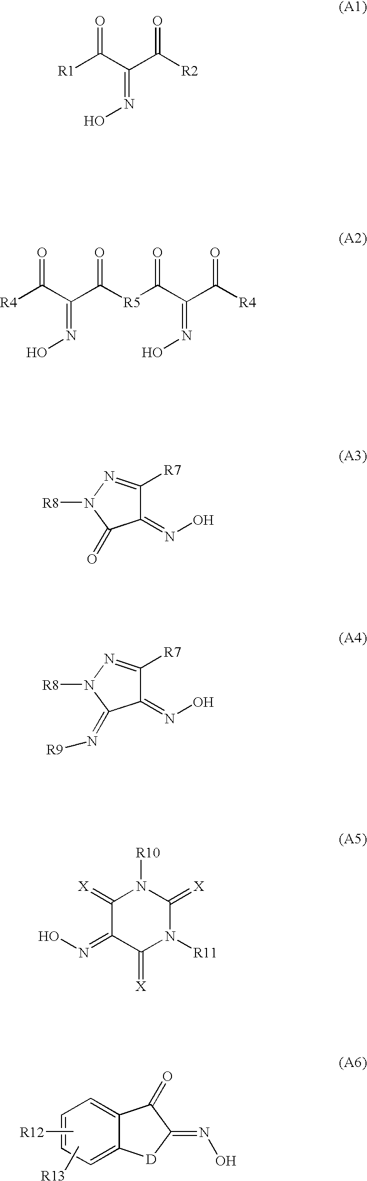 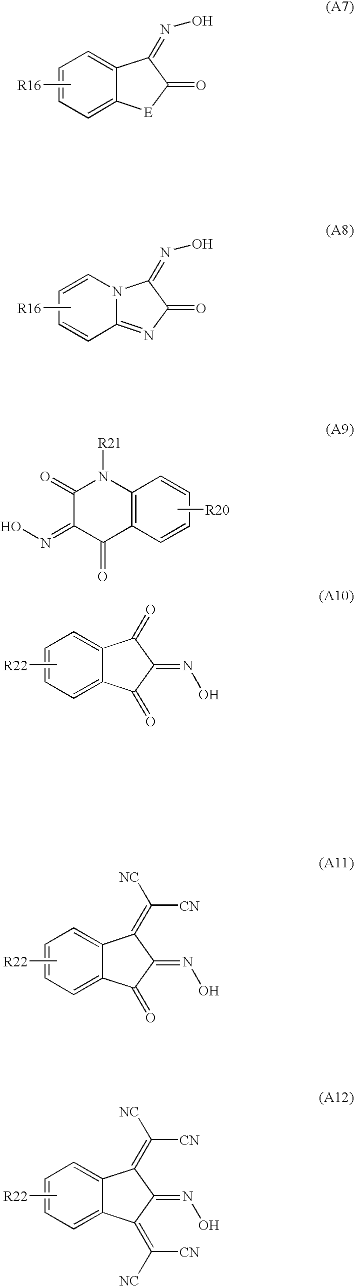 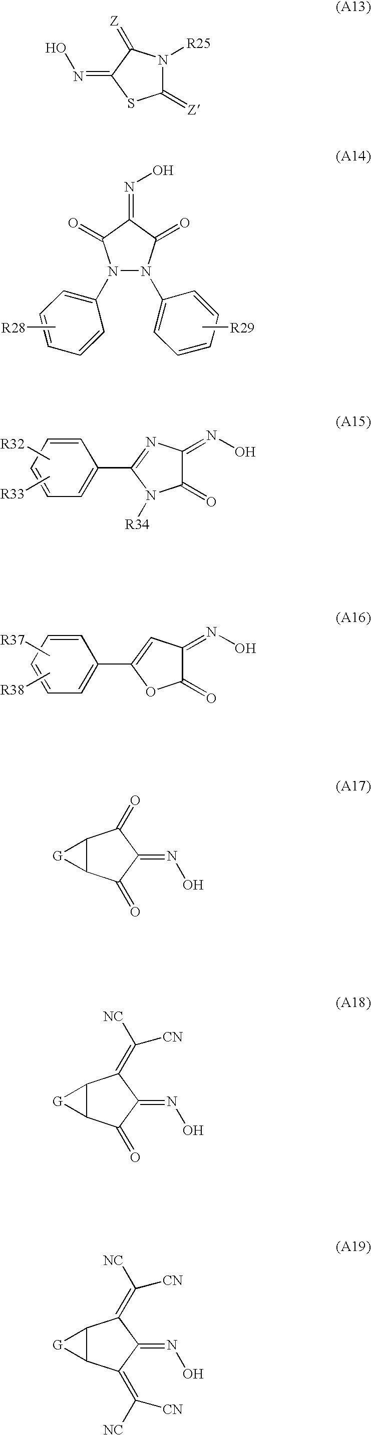 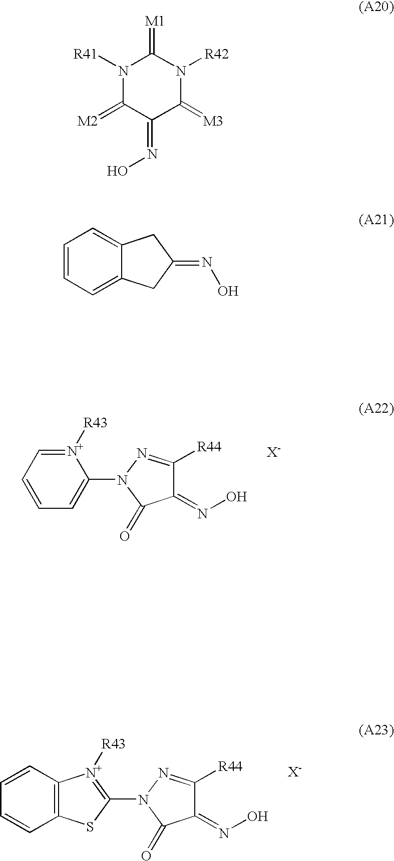
in which
R1 is a cyano group, a substituted or unsubstituted aryl group or a substituted or unsubstituted, saturated or unsaturated heterocycle or a group —C—(O)—R3, where R3 is a hydrogen atom or a C₁-C₆-alkyl group;
R2 is a cyano group, a substituted or unsubstituted C₁-C₆-alkyl group, a C₃-C₆-cycloalkyl group, a substituted or unsubstituted aryl group or a substituted or unsubstituted aminoaryl group or a substituted or unsubstituted, saturated or unsaturated heterocycle;
R4 is a cyano group, a substituted or unsubstituted aryl group, a substituted or unsubstituted, saturated or unsaturated heterocycle or a group —C—(O)—R6, where R6 is a hydrogen atom or a C₁-C₆-alkyl group;
R5 is a substituted or unsubstituted C₂-C₆-alkyl group, a C₃-C₆-cycloalkyl group, a substituted or unsubstituted aryl group, a substituted or unsubstituted aminoaryl group, a substituted or unsubstituted, saturated or unsaturated heterocycle;
in which R7 and R8 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group, a substituted or unsubstituted aminoaryl group, where R7 may be a C₁-C₆-alkoxy group;
R9 is a hydrogen atom or a substituted or unsubstituted C₁-C₆-alkyl group;
R10 and R11 may be identical or different and, independently of one another, are a substituted or unsubstituted aryl group, an aryl-C₁-C₄-alkyl group, a C₁-C₆-alkyl group, a C₃-C₆-cycloalkyl group, a C₂-C₆-alkylene group or a bicycle bonded via the radicals R10 and R11;
X is an oxygen atom or a sulphur atom;
D is an oxygen atom, a sulphur atom, a sulphoxyl group, a sulphonyl group or a group N—R₁₂ₐ, where R₁₂ₐ is a hydrogen atom or a substituted or unsubstituted C₁-C₆-alkyl group;
and R12 and R13, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR14R15. where R14 and R15, independently of one another, are a hydrogen atom or a C₁-C₆-alkyl group;
E is an oxygen atom, a sulphur atom or a group 1NR7, where R17 is a hydrogen atom or a substituted or unsubstituted C₁-C₆-alkyl group;
R16 is a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR18R19, where R18 and R19, independently of one another, are a hydrogen atom or a C₁-C₆-alkyl group;
R20 is a hydrogen atom, a halogen atom, a hydroxyl group, a nitro group, a C₁-C₆-alkoxy group or a substituted or unsubstiturted C₁-C₆-alkyl group;
R21 is a hydrogen atom or a C₁-C₄-alkyl group;
R22 is a hydrogen atom, a halogen atom, a hydroxyl group, a nitro group, a C₁-C₆-alkyl group, a C₁-C₆-alkoxy group, a carboxyl group, a carboxamide group, a cyano group or an amino group —NR23R24, where R23 and R24, independently of one another, are a hydrogen atom or a C₁-C₆-alkyl group;
Z is an oxygen atom or a group —NR26, where R26 is a hydrogen atom or a C₁-C₆-alkyl group;
Z′ is a sulphur atom or a group —NR27, where R27 is a hydrogen atom or a C₁-C₆-alkyl group;
R25 is a hydrogen atom, a C₁-C₆-alkyl group or a C₁-C₄-carboxyalkyl group;
R28 and R29 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR30R31, where R30 and R31, independently of one another, are a hydrogen atom or a C₁-C₆-alkyl group;
R32 and R33 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR35R36, where R35 and R36, independently of one another, are a hydrogen atom or a C!-C₆-alkyl group:
R34 is a hydrogen atom or a C₁-C₆-alkyl group;
R37 and R38 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR39R40, where R39 and R40, independently of one another, are a hydrogen atom or a C₁-C₆-alkyl group;
G is a fused, substituted or unsubstituted aromatic or heteroaromatic ring to which a further aromatic or heteroaromatic ring may additionally be fused;
R41 and R42 may be identical or different and, independently of one another, are a hydrogen atom or a substituted or unsubstituted C₁-C₆-alkyl group;
M1 is an oxygen atom, a sulphur atom or an NH group;
and M2 and M3 may be identical or different and, independently of one another, are an oxygen atom or an NH group;
R43 is a substituted or unsubstituted C₁-C₆-alkyl group; R44 is a substituted or unsubstituted aryl group or an aryl-C₁-C₄-alkyl group, a substituted or unsubstituted, saturated or unsaturated heterocycle, a substituted or unsubstituted C₁-C₆-alkyl group, an acetyloxy group, a C₃-C₆-cycloalkyl group, a C₁-C₆-alkoxy group or a substituted or unsubstituted aminoaryl group; and
X⁻ is a counterion;
and c) at least one reactive carbonyl compound of the formula B1-B17; 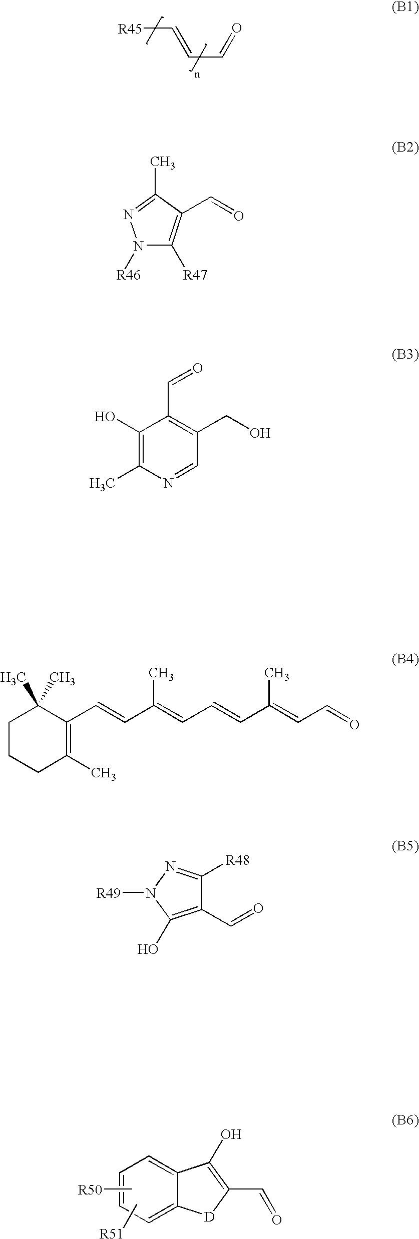 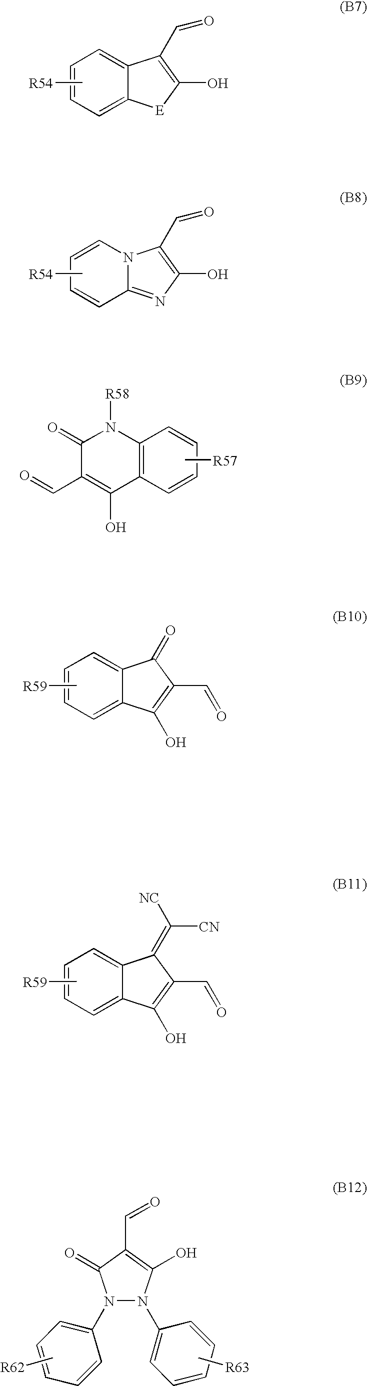 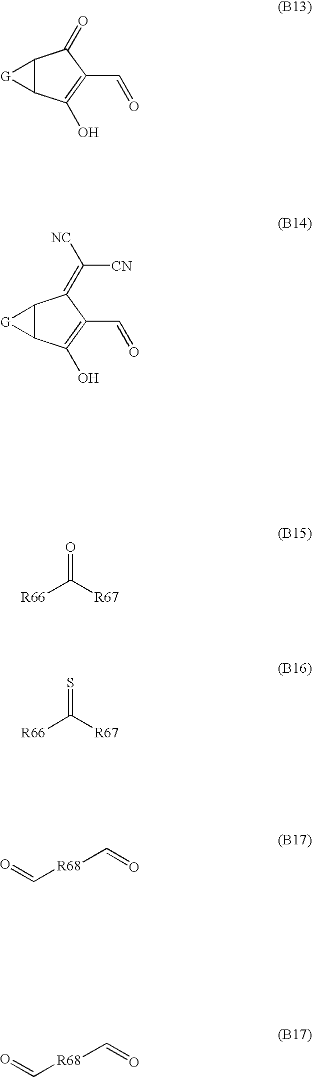
in which
R45 is a substituted or unsubstituted aryl group to which a further aromatic ring may optionally also be fused, a substituted or unsubstituted, saturated or unsaturated heterocycle;
n is 0, 1,2 or 3;
R46 is a C₁-C₆-alkyl group, a substituted or unsubstituted aryl radical or a substituted or unsubstituted, saturated or unsaturated heterocycle;
R47 is a pyrrole radical, an imidazole radical, a pyrazole radical, a indole radical, a pyrrolidine radical, a morpholine radical, a dimethylamine radical, a phenol radical or a thiophenol radical, where these radicals are in each case bonded to the general formula B2 via the heteroatom;
R48 and R49 may be identical or different and, independently of one another, are a substituted or unsubstituted aryl group, an aryl-C₁-C₄-alkyl group, a substituted or unsubstituted, saturated or unsaturated heterocycle, a substituted or unsubstituted C₁-C₆-alkyl group, an acetyloxy group, a C₃-C6-cycloalkyl group or a substituted or unsubstituted aminoaryl group, and R48 may be a C₁-C₆-alkoxy group;
D has the abovementioned meaning;
R50 and R51 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR52R53, where R52 and R53, independently of one another, are a hydrogen atom or a C₁-C₆-alkyl group;
E has the abovementioned meaning;
R54 is a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR55R56, where R55 and R56, independently of one another, are a hydrogen atom or a C₁-C₆-alkyl group;
R57 is a hydrogen atom, a halogen atom, a hydroxyl group, a nitro group, a C₁-C₆-alkoxy group or a substituted or unsubstituted C₁-C₆-alkyl group;
R58 is a hydrogen atom or a C₁-C₄-alkyl group;
R59 is a hydrogen atom, a halogen atom, a hydroxyl group, a nitro group, a C₁-C₆-alkyl an amino group —NR60R61, where R60 and R61, independently of one another, are a hydrogen atom or a C₁-C₆-alkyl group;
R62 and R63 may be identical or different and, independently of one another, are a hydrogen atom, a fluorine atom, a chlorine atom, a bromine atom, a hydroxyl group, a nitro group, a C₁-C₆-alkyl group, a C₁-C₆-alkoxy group, a carboxamide group, a sulphonamide group, a carboxyl group, a C₁-C₄-acyl group, a cyano group or an amino group —NR64R65, where R64 and R65, independently of one another, are a hydrogen atom or a C₁-C₆-alkyl group; in which G has the abovementioned meaning;
R66 and R67 may be identical or different and, independently of one another, are a C₁-C₆-alkyl group or a substituted or unsubstituted aryl group; and
R68 is a substituted or unsubsituted C₁-C₆-alkyl group, a C₁-C₆-alkylene group or a substituted or unsubstituted aryl group.

2. An agent according to claim 1, wherein the oxime compound of the general formula A1-A23 is selected from the group consisting of diethyl 2-(hydroxyimino)malonate, 1H-indene-1,2,3-trione 2-oxime, (2Z)-5-(dimethylamino)-1H-indene-1,2,3-trione 2-oxime, (2Z)-4,5-dimethoxy-1H-indene-1,2,3-trione 2-oxime, (2Z)-5-methoxy-1H-indene-1,2,3-trione 2-oxime, (2Z)-5-nitro-1H-indene-1,2,3-trione 2-oxime, (2Z)4,6-dichloro-1H-indene-1,2,3-trione 2-oxime, (6Z)-5H-cyclopenta[c]pyridine-5,6,7-trione 6-oxime, (6Z)-5H-cyclopenta[b]pyridine-5,6,7-trione 6-oxime, (2E)-1H-cyclopenta[a]-naphthalene-1,2,3-trione 2-oxime, 1H-cyclopenta[b]quinoxaline-1,2,3-trione 2-oxime, (2Z)-4-methyl-4,8b-dihydrocyclopenta[b]indole-1,2,3(3aH)-trione 2-oxime, (5E)-4H-cyclopenta[b]thiophene-4,5,6-trione 5-oxime, (2Z)-5-methyl-1H-indene-1,2,3-trione 2-oxime, (2E)-4,6-dichloro-1H-indene-1,2,3-trione 2-oxime, 4,7-dichloro-1H-indene-1,2,3-trione 2-oxime, (2Z)-1H-cyclopenta[b]quinoline-1,2,3-trione 2-oxime, (2Z)-1-benzothiophene-2,3-dione 2-oxime 1,1-dioxide, (2Z)-1-benzothiophene-2,3-dione 2-oxime, (2Z)-l-benzofuran-2,3-dione 2-oxime, (3E)-imidazo[1,2-a]pyridine-2,3-dione 3-oxime, (3Z)-1H-indole2,3-dione 3-oxime, (3Z)-1-methyl-1H-indole-2,3-dione 3-oxime, (3Z)-6-chloro-1-methyl-1H-indole-2,3-dione 3-oxime, (3E)-7-chloroimidazo[1,2-a]pyridine-2,3-dione 3-oxime, 1,2-diphenyl-3,4,5-pyrazolidinetrione-4-oxime, 1H-cyclopenta[b]naphthalene-1,2,3-trione 2-oxime, 2-[(2E)-2-(hydroxyimino)-3-oxo-2,3-dihydro-1H-indene-1-ylidene]malononitrile, (4Z)-3-methyl-1-phenyl-1H-pyrazole-4,5-dione 4-oxime, 1,3-diethyl-2-thioxodihydro-4,5,6(1H)-pyrimidinetrione 5-oxime, (5Z)-2-thioxo-1,3-thiazolidine-4,5-dione 5-oxime, (⁴Z)-3-methyl-1-(4-nitrophenyl)-1H-pyrazole-4,5-dione 4-oxime, 1,3-dihydro-2H-inden-2-one oxime, 4,5,6,7-tetrachloro-1H-indene-1,2,3-trione 2-oxime, 2-[(4E)-4-(hydroxyimino)-3-methyl-5-oxo-4,5-dihydro-1H-pyrazol-1-yl]-1-methylpyridinium methylsulphate and 2-[(⁴E)-4-(hydroxyimino)-3-methyl-5-oxo-4,5-dihydro-1H-pyrazol-1-yl]-3-methyl-1,3-benzothiazol-3-ium methylsulphate.

3. An agent according to claim 1, wherein the reactive carbonyl compound of the general formulae B1-B17 is selected from the group consisting of benzaldehyde, 4-methoxybenzaldehyde, 4-hydroxybenzaldehyde, 3,4-dimethoxybenzaldehyde, 2,5-dimethoxybenzaldehyde, 3-hydroxy-4-methoxybenzaldehyde, 4-nitrobenzaldehyde, 4-hydroxy-3-methoxy-benzaldehyde, ⁴-(dimethylamino)benzaldehyde, (2E)-3-[4-(dimethylamino)phenyl]-2-propenal, 2,4,6-trihydroxybenzaldehyde, 3-hydroxy-5-(hydroxymethyl)-2-methylisonicotine aldehyde, all-trans-retinal, 3,5-dioxo-1,2-diphenyl-4-pyrazolidinecarbaldehyde, (2E,4E)-5-phenyl-2,4-pentadienal, pentanedial, 4-(methylsulphanyl)benzaldehyde, 1-naphthaldehyde, (2E)-3-(4-nitrophenyl)-2-propenal, 3-phenoxybenzaldehyde, terephthalaldehyde, 3-hydroxy-1-oxo-1H-cyclopenta[a]-naphthalene-2-carbaldehyde, 3-hydroxy-1-oxo-1H-cyclopenta[b]-quinoxaline-2-carbaldehyde, 3-hydroxy-4-methyl-1-oxo-1,3a,4,8b-tetrahydrocyclopenta[b]indole-2-carbaldehyde, 4-hydroxy-6-oxo-6H-cyclopenta[b]thiophene-5-carbaldehyde, 3-hydroxy-5-nitro-1-oxo-1H-indene-2-carbaldehyde, 4,6-dichloro-3-hydroxy-1-oxo-1H-indene-2-carbaldehyde, 7-hydroxy-5-oxo-5H-cyclopenta[c]pyridine-6-carbaldehyde, 7-hydroxy-5-oxo-5H-cyclopenta[b]pyridine-6-carbaldehyde, 3-hydroxy-1-oxo-1H-indene-2-carbaldehyde, 5-(dimethylamino)-3-hydroxy-1-oxo-1H-indene-2-carbaldehyde, 3-hydroxy-4,5-dimethoxy-1-oxo-1H-indene-2-carbaldehyde, 3-hydroxy-5-methoxy-1-oxo-1H-indene-2-carbaldehyde, 3-methyl-S -(4-morpholinyl)-1-phenyl-1H-pyrazole-4-carbaldehyde, 3-methyl-1-phenyl-5-(1H-pyrrol-1-yl)-1H-pyrazole-4-carbaldehyde, 3-methyl-1-phenyl-5-( 1-pyrrolidinyl)-1H-pyrazol-4-carbaldehyde, 3-methyl-1-(2-pyridinyl)-5-(1-pyrrolidinyl)-1H-pyrazole-4-carbaldehyde, 3-methyl-1-(2-pyridinyl)-5-(1H-pyrrol-1-yl)-1H-pyrazole-4-carbaldehyde, 4-methoxy-1-naphthaldehyde, 1H-indole-3-carbaldehyde, bis[4-(dimethylamino)-phenyl]methanethione, 2,4-dihydroxybenzaldehyde, 1-methyl-1H-indole-3-carbaldehyde and 3,4-dihydroxybenzaldehyde.

4. An agent according to claim 1, comprising the oxime compound of the general formula A1-A23 and the reactive carbonyl compound of the general formula B1-B17 in each case in an amount of from 0.01 to 10 per cent by weight.

5. An agent according to claim 1, additionally comprising one or more customary direct dyes selected from the group consisting of acidic and basic dyes, nitro dyes, azo dyes, anthraquinone dyes and triphenylmethane dyes.

6. An agent according to claim 1, wherein the agent is a hair colorant.

7. An agent according to claim 1, additionally comprising at least one natural or synthetic polymer or modified polymer of natural origin customary for cosmetic compositions, and is in the form of a tinting setting composition or colour setting composition.

8. A method of colouring hair in which a colorant according to claim 1 is applied to the hair in an amount adequate for the hair colouring, about 30 to 120 grams, the hair colorant is left to act at about 15 to 45 degrees Celsius for about 1 to 60 minutes, and the hair is then thoroughly rinsed with water, optionally washed with a shampoo and/or after-treated with a hair-conditioning composition and finally dried.
